(19) United States
(12) Patent Application Publication
(10) Pub. No.: US 2016/0233545 A1
(43) Pub. Date: Aug. 11, 2016

(54) ELECTROLYTE ADDITIVE AND USE THEREOF

(71) Applicant: Ningde Amperex Technology Limited, Ningde City (CN)

(72) Inventors: Peipei CHEN, Ningde City (CN); Bing Long, Ningde City (CN); Kefei Wang, Ningde City (CN)

(73) Assignee: Ningde Amperex Technology Limited, Ningde City (CN)

(21) Appl. No.: 14/963,096

(22) Filed: Dec. 8, 2015

(30) Foreign Application Priority Data
Feb. 6, 2015 (CN) 201510063456.7

(51) Int. Cl. H01M 10/0567; C07D 495/04; H01M 10/0525

(52) U.S. Cl. CPC H01M 10/0567; H01M 10/0525; C07D 495/04; H01M 2300/0025; H01M 2220/30

(57) ABSTRACT

The present application discloses an electrolyte additive comprising a thiophene compound. When used in a lithium ion battery, the additive can inhibit a sustained electrochemical oxidation reaction between an electrolyte and a positive electrode material, thereby improving the electrical conductivity of the positive electrode material, enhancing the cycle performance and high-current discharge performance of the battery, and improving the safety performance and rate performance of the battery.

### TECHNICAL FIELD

The present application belongs to the field of batteries, and particularly relates to a non-aqueous electrolyte and a lithium ion battery using the same.

### BACKGROUND

With the rapid development of the information age, the demands for electronic products are increasing year by year. Lithium ion batteries are characterized by a high energy density, no memory effect, a high operating voltage, a broad temperature application range and the like, and therefore have currently been widely applied to electronic products such as mobile phones, laptop computers and cameras, and are gradually replacing traditional Ni—Cd and MH—Ni batteries to become major chemical power sources.

With the expanding demands in electronic product markets and the development of energy storage devices, people have ever-increasing requirements on lithium ion batteries. It is urgently desired to develop a lithium ion battery having the advantages of high energy, a long service life, rapid charging and discharging, and high safety. Positive electrode materials have always been considered as an important factor for restricting the development of lithium ion batteries. The reasons are as follows: when a lithium ion battery is charged or discharged, a transition metal in a positive electrode active material (lithium composite oxides such as lithium cobaltate, lithium manganate and a ternary material) exhibits a high valence state which causes an electrolyte to be easily oxidized, thereby severely affecting the service life and safety of the battery. Accordingly, the reduction or inhibition of the reaction activity of positive electrode materials is the key to further development of lithium ion batteries. The current major solution is to add a film-forming additive to an electrolyte. Common film-forming additives include vinylene carbonate (VC), fluoroethylene carbonate (FEC), propylene sulfite (PS), ethylene sulfite (ES), lithium bis(oxalate)borate (LiBOB), and the like. These additives can form a film on a positive electrode, but would cause increased interface impedance and result in reduced dynamic performance of lithium ion migration and diffusion in a battery, thereby attenuating the rate and cycle performance of the battery.

Accordingly, the development of an additive capable of effectively acting on a positive electrode surface has become an important research direction.

### SUMMARY

According to one aspect of the present application, there is provided an electrolyte additive, wherein a protective film may be formed on a positive electrode surface of a lithium ion battery by use of the additive, and the protective film not only has good electrical conductivity, but also inhibits a side reaction between a positive electrode active material and an electrolyte, thereby improving the cycling stability and safety performance of the battery, and enhancing the rate performance of the battery.

Two electron-donating oxygen atoms are introduced on a thiophene ring such that its oxidation potential is about 4.0 V (with respect to Li/Li⁺), which is applicable to a lithium ion battery at more than 4.0 V. When the voltage of a lithium ion battery reaches the oxidation potential of an additive, a double bond and a cyclic structure contained in a molecule of the additive are subjected to an oxidation reaction to generate a product adhered on a positive electrode surface, thereby forming a layer of a protective film. Meanwhile, a sulfur-containing structure is oxidized to generate a sulfonate substance which is capable of allowing an interfacial film to be more compact and more uniform, and capable of inhibiting a sustained reaction between a positive electrode active material and an electrolyte, thereby improving the safety performance of the battery. The interfacial film generated on the positive electrode surface by the additive has better ionic and electronic conduction capability, thereby facilitating the battery to obtain good dynamics performance.

The electrolyte additive is characterized by comprising a thiophene compound selected from at least one of a compound having a chemical structural formula as shown by Formula I and a compound having a chemical structural formula as shown by Formula II:

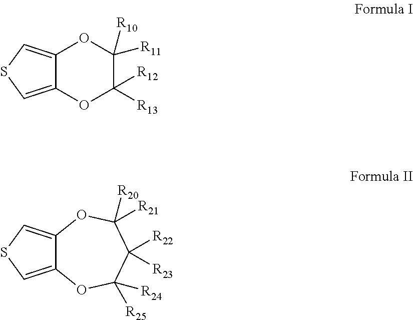

wherein R₁₀, R₁₁, R₁₂, R₁₃, R₂₀, R₂₁, R₂₂, R₂₃, R₂₄ and R₂₅ are respectively independently selected from hydrogen, fluoro, chloro, bromo, iodo, nitro, a sulfonic acid group, hydrocarbyl having 1-10 carbon atoms, and a group having 1-10 carbon atoms and containing at least one element selected from fluorine, chlorine, bromine, iodine, nitrogen, oxygen and sulfur.

The hydrocarbyl having 1-10 carbon atoms is formed by loss of any hydrogen atom on a molecule of a hydrocarbon compound having 1-10 carbon atoms, wherein the hydrocarbon compound is selected from saturated or unsaturated hydrocarbons, including but not limited to alkanes, cycloalkanes, alkenes, alkynes and aromatic hydrocarbons.

The group having 1-10 carbon atoms and containing at least one element selected from fluorine, chlorine, bromine, iodine, nitrogen and oxygen is formed by loss of any hydrogen atom on a molecule of a compound having 1-10 carbon atoms and containing at least one element selected from fluorine, chlorine, bromine, iodine, nitrogen and oxygen. For example, a nitrile group is formed by loss of one hydrogen atom in hydrocyanic acid having one carbon atom and containing nitrogen; and nitroethyl is formed by loss of one hydrogen atom in nitroethane having two carbon atoms and containing nitrogen and oxygen, and the like.

Preferably, R₁₀, R₁₁, R₁₂, R₁₃, R₂₀, R₂₁, R₂₂, R₂₃, R₂₄ and R₂₅ are respectively independently selected from hydrogen, fluoro, chloro, bromo, iodo, nitro, cyano, carboxyl, a sulfonic acid group, hydrocarbyl having 1-10 carbon atoms, a group having 1-10 carbon atoms and containing at least one group selected from fluoro, chloro, bromo, iodo, nitro and a sulfonic acid group, and a group having 2-10 carbon atoms and containing at least one group selected from cyano, carboxyl and alkoxy.

Preferably, R₁₀, R₁₁, R₁₂, R₁₃, R₂₀, R₂₁, R₂₂, R₂₃, R₂₄ and R₂₅ are respectively independently selected from hydrogen, hydrocarbyl having 1-3 carbon atoms, and a group having 1-3 carbon atoms and containing fluorine and/or alkoxy.

Preferably, the thiophene compound is selected from at least one of 2-methyl-2,3-dihydrothieno[3,4-b][1,4]dioxane, 2-methoxymethyl-2,3-dihydrothieno[3,4-b][1,4]dioxane, 3,4-propylenedioxythiophene, 3,4-ethylenedioxythiophene, 3,4-(2,2-dimethylpropylenedioxy)thiophene and 3,4-(2,2-diethylpropylenedioxy)thiophene.

The 2-methyl-2,3-dihydrothieno[3,4-b][1,4]dioxane has a structural formula as shown by Formula III:

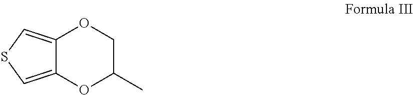

The 3,4-propylenedioxythiophene has a structural formula as shown by Formula IV:

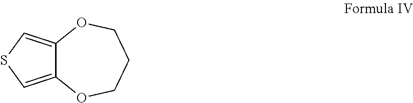

The 3,4-ethylenedioxythiophene has a structural formula as shown by Formula V:

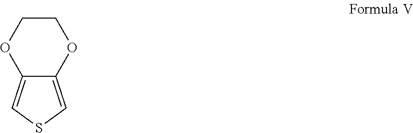

The 2-methoxymethyl-2,3-dihydrothieno[3,4-b][1,4]dioxane has a structural formula as shown by Formula VI:

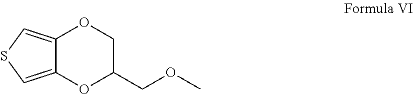

The 3,4-(2,2-dimethylpropylenedioxy)thiophene has a structural formula as shown by Formula VII:

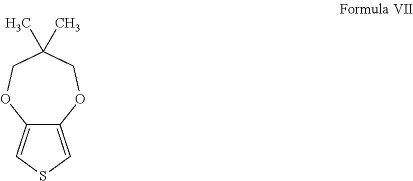

The 3,4-(2,2-diethylpropylenedioxy)thiophene has a structural formula as shown by Formula VIII:

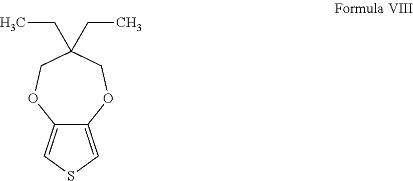

Preferably, the additive further comprises at least one of a film former of a solid electrolyte interface film, a flame retardant, an overcharge protection agent and a stabilizer.

Further preferably, the additive further comprises a film former of a solid electrolyte interface film.

In the present application, the term “solid electrolyte interface” may be abbreviated as SEI.

Preferably, the film former of a solid electrolyte interface film is selected from at least one of vinylene carbonate (abbreviated as VC), fluoroethylene carbonate (abbreviated as FEC), chloroethylene carbonate (abbreviated as ClEC), propane sultone (abbreviated as PS), butane sultone (abbreviated as BS) and adiponitrile (abbreviated as ADN).

The thiophene compound has a mass percent content of 10%-100% in the additive. Preferably, the thiophene compound has a mass percent content of 50%-100% in the additive.

The film former of a solid electrolyte interface film has a mass percent content of 0%-50% in the additive.

As a preferred embodiment, the additive consists of a thiophene compound and a film former of a solid electrolyte interface film.

According to another aspect of the present application, there is provided an electrolyte for a lithium ion battery, which is characterized by containing at least one of the additives.

The electrolyte for a lithium ion battery comprises an organic solvent, a lithium salt and an additive.

Preferably, the thiophene compound has a mass percent content of 0.1%-10% in the electrolyte. Further preferably, the upper limit and the lower limit of the mass percent content of the thiophene compound in the electrolyte are respectively selected from 10%, 8% and 6%, and from 0.1%, 0.5%, 1% and 3%. Still further preferably, the thiophene compound has a mass percent content of 1%-8% in the electrolyte.

Preferably, the film former of a solid electrolyte interface film has a mass percent content of 0.1%-10% in the electrolyte. Further preferably, the upper limit and the lower limit of the mass percent content of the film former of a solid electrolyte interface film in the electrolyte are respectively selected from 10%, 8% and 6%, and from 0.1%, 0.5%, 1% and 3%. Still further preferably, the film former of a solid electrolyte interface film has a mass percent content of 1%-8% in the electrolyte.

Preferably, the additive has a mass percent content of 0.1%-20% in the electrolyte.

Preferably, the organic solvent is selected from at least one of ethylene carbonate, propylene carbonate, dimethyl carbonate, diethyl carbonate, dipropyl carbonate, ethyl methyl carbonate, methyl formate, ethyl formate, ethyl propionate, propyl propionate, methyl butyrate, ethyl acetate, acid anhydride, N-methylpyrrolidone, N-methylformamide, N-methylacetamide, acetonitrile, sulfolane, dimethyl sulfoxide, ethylene sulfite, propylene sulfite, dimethyl sulfide, diethyl sulfite, dimethyl sulfite, tetrahydrofuran, a fluorine-containing cyclic organic ester and a sulfur-containing cyclic organic ester.

Further preferably, the organic solvent is selected from at least two of ethylene carbonate, propylene carbonate, dimethyl carbonate, diethyl carbonate, dipropyl carbonate, ethyl methyl carbonate, methyl formate, ethyl formate, ethyl propionate, propyl propionate, methyl butyrate, ethyl acetate, acid anhydride, N-methylpyrrolidone, N-methylformamide, N-methylacetamide, acetonitrile, sulfolane, dimethyl sulfoxide, ethylene sulfite, propylene sulfite, dimethyl sulfide, diethyl sulfite, dimethyl sulfite, tetrahydrofuran, a fluorine-containing cyclic organic ester and a sulfur-containing cyclic organic ester.

The organic solvent has a mass percent content of 60%-90% in the electrolyte.

Preferably, the lithium salt is optionally selected from at least one of an organic lithium salt or an inorganic lithium salt.

Preferably, the lithium salt is selected from at least one of LiPF₆, LiBF₄, LiTFSI, LiClO₄, LiAsF₆, LiBOB, LiDFOB, LiTFOB, LiN(SO₂R_{F})₂ and LiN(SO₂F)(SO₂R_{F}), wherein a substituent R_{F}=CₙF₂ₙ₊₁ is saturated perfluoroalkyl, n is an integer from 1 to 10, and accordingly 2n+1 is an integer greater than zero.

Preferably, the lithium salt has a concentration of 0.5 mol/L-2 mol/L in an electrolyte for a lithium ion secondary battery. Further preferably, the lithium salt has a concentration of 0.9 mol/L-1.3 mol/L in the electrolyte.

As a preferred embodiment, the electrolyte consists of a non-aqueous organic solvent, a lithium salt and an additive.

According to still another aspect of the present application, there is provided a lithium ion battery, which is characterized by containing at least one of the additives.

The lithium ion battery comprises a positive electrode current collector and a positive electrode membrane coated on the positive electrode current collector, a negative electrode current collector and a negative electrode membrane coated on the negative electrode current collector, a diaphragm and an electrolyte.

The electrolyte contains at least one of the additives.

The positive electrode membrane comprises a positive electrode active material, a binder and a conductive agent.

The negative electrode membrane comprises a negative electrode active material, a binder and a conductive agent.

The positive electrode active material is optionally selected from at least one of lithium cobaltate (LiCoO₂), lithium nickelate (LiNiO₂), lithium iron phosphate (LiFePO₄), lithium manganate (LiMnO₂), a ternary material LiNiₓA_{y}B_{(1-x-y)}O₂ (in which, A and B are independently selected from at least one of Co, Al and Mn, A is different from B, 0<x<1, and 0<y<1), olivine-type LiMPO₄ (in which, M is selected from at least one of Co, Ni, Fe, Mn and V), and Li₁₋ₓ(A_{y}B_{z}C_{1-y-z})O₂ (in which, 0≦x<1, 0≦y<1, 0≦z<1, and A, B and C are independently selected from at least one of Co, Ni, Fe and Mn).

The negative electrode active material is selected from, but not limited to at least one of metallic lithium, natural graphite, artificial graphite, mesocarbon microbeads (abbreviated as MCMB), hard carbon, soft carbon, silicon, a silicon-carbon complex, a Li—Sn alloy, a Li—Sn—O alloy, Sn, SnO, SnO₂, lithiated TiO₂—Li₄Ti₅O₁₂ with a spinel structure, and a Li—Al alloy.

The present application achieves at least the following beneficial effects:

(1) When used in a lithium ion battery, the electrolyte additive provided by the present application is capable of significantly improving the rate discharge performance of the lithium ion battery, reducing the internal resistance of the lithium ion battery, and enhancing the cycle performance of the lithium ion battery at high temperature.

(2) The lithium ion battery provided by the present application has excellent rate discharge performance.

(3) The lithium ion battery provided by the present application has lower internal resistance.

(4) The lithium ion battery provided by the present application has excellent high-temperature cycle performance.

### DETAILED DESCRIPTION

The present application is hereinafter described in detail with reference to examples, but the present application is not limited to these examples.

In the examples, 2-methyl-2,3-dihydrothieno[3,4-b][1,4]dioxane and 3,4-propylenedioxythiophene are commercially available from Sigma-Aldrich (China).

A binder polyvinylidene fluoride (abbreviated as PVDF) is commercially available from Polyfluoro Factory of Zhejiang Juhua Joint-stock Co., Ltd., a thickener sodium carboxymethyl cellulose (abbreviated as CMC) is commercially available from Quanzhou Zhongxin Industry Co., Ltd., conductive carbon black Super-P is commercially available from Tianjin Jindadi Chemical Co., Ltd., and an adhesive styrene butadiene rubber (abbreviated as SBR) is commercially available from Shanghai Jedo Chemical Co., Ltd.

The electrochemical performance of batteries is determined by a BTS series battery testing cabinet from Shenzhen Neware Technology Co., Ltd.

### Example 1

### Preparation of Positive Electrode Sheet P1^{#}

A positive electrode active material lithium cobaltate (molecular formula: LiCoO₂), a conductive agent (a carbon nanotube CNT had a mass percent content of 6% and conductive carbon black had a mass percent content of 94% in the conductive agent), and a binder polyvinylidene fluoride (abbreviated as PVDF, polyvinylidene fluoride had a mass percent content of 7% in the binder) were uniformly dispersed into a solvent N-methylpyrrolidone (abbreviated as NMP) to prepare a positive electrode slurry. The positive electrode slurry had a solid content of 77 wt %, and solid ingredients comprised 98.26 wt % of lithium cobaltate, 0.9 wt % of PVDF and 0.84 wt % of the conductive agent. The positive electrode slurry was uniformly coated on a positive electrode current collector aluminum foil having a thickness of 12 μm, wherein the coating amount at a single side was 0.0215 g/cm². Subsequently, the resulting material was oven-dried at 85° C., then subjected to chill pressing, edge trimming, piece cutting and slitting, and then dried for 4 h under vacuum conditions at 85° C., and a lug was welded to obtain a positive electrode sheet recorded as P1^{#}.

### Preparation of Negative Electrode Sheet N1^{#}

A negative electrode active material artificial graphite, a thickener sodium carboxymethyl cellulose (abbreviated as CMC, sodium carboxymethyl cellulose had a mass percent content of 1.5%), and an adhesive styrene butadiene rubber (abbreviated as SBR, styrene butadiene rubber had a mass percent content of 40% in the adhesive) were uniformly mixed in deionized water to prepare a negative electrode slurry. The negative electrode slurry had a solid content of 54 wt %, and solid ingredients comprised 97.8 wt % of artificial graphite, 1.1 wt % of CMC and 1.1 wt % of SBR. The negative electrode slurry was uniformly coated on a negative electrode current collector copper foil having a thickness of 8 μm, wherein the coating amount was 0.0107 g/cm². Subsequently, the resulting material was oven-dried at 85° C., then subjected to chill pressing, edge trimming, piece cutting and slitting, and then dried for 4 h under vacuum conditions at 110° C., and a lug was welded to obtain a negative electrode sheet recorded as N1^{#}.

### Preparation of Electrolyte L1^{#}

In a drying room, ethylene carbonate (abbreviated as EC) and ethyl methyl carbonate (abbreviated as EMC) were evenly mixed by a volume ratio of EC to EMC=3:7 to obtain an organic solvent. A conductive lithium salt LiPF₆ and an additive 2-methyl-2,3-dihydrothieno[3,4-b][1,4]dioxane (having a structural formula as shown by Formula III, and abbreviated as Formula III in Table 1) were added to the organic solvent to obtain a solution in which the additive 2-methyl-2,3-dihydrothieno[3,4-b][1,4]dioxane had a mass percent content of 1% and LiPF₆ had a concentration of 1 mol/L, i.e., an electrolyte recorded as L1^{#}.

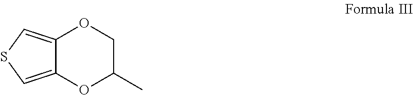

### Preparation of Lithium Ion Secondary Battery C1^{#}

A 12 μm polypropylene film served as a diaphragm.

The positive electrode sheet P1^{#}, the diaphragm and the negative electrode sheet N1^{#} were sequentially stacked with the diaphragm placed between positive and negative electrodes for the purpose of isolation, and then wound to obtain a square bare cell having a thickness of 3 mm, a width of 35 mm and a length of 95 mm. The bare cell was loaded into an aluminum foil packaging bag, baked for 10 h under vacuum conditions at 75° C., then injected with the electrolyte L1^{#}, subjected to vacuum encapsulation, kept still for 24 h, then charged to 4.35 V at a constant current of 0.1 C (160 mA), then charged at a constant voltage of 4.35 V until the current decreased to 0.05 C (100 mA), then discharged to 3.0 V at a constant current of 0.1 C (200 mA) (charging and discharging were repeated twice), and finally charged to 3.85 V at a constant current of 0.1 C (200 mA), thereby completing the preparation of a lithium ion secondary battery, wherein the resulting lithium ion secondary battery was recorded as C1^{#}.

### Example 2

### Preparation of Electrolyte L2^{#}

This preparation method was the same as that of the electrolyte L1^{#}, except that the additive 2-methyl-2,3-dihydrothieno[3,4-b][1,4]dioxane had a mass percent content of 0.1% instead of 1% and the resulting electrolyte was recorded as L2^{#}.

### Preparation of Lithium Ion Secondary Battery C2^{#}

This preparation method was the same as that of the lithium ion secondary battery C1^{#}, except that the electrolyte L2^{#} was used instead and the resulting lithium ion secondary battery was recorded as C2^{#}.

### Example 3

### Preparation of Electrolyte L3^{#}

This preparation method was the same as that of the electrolyte L1^{#}, except that the additive 2-methyl-2,3-dihydrothieno[3,4-b][1,4]dioxane had a mass percent content of 3% instead of 1% and the resulting electrolyte was recorded as L3^{#}.

### Preparation of Lithium Ion Secondary Battery C3^{#}

This preparation method was the same as that of the lithium ion secondary battery C1^{#}, except that the electrolyte L3^{#} was used instead and the resulting lithium ion secondary battery was recorded as C3^{#}.

### Example 4

### Preparation of Electrolyte L4^{#}

This preparation method was the same as that of the electrolyte L1^{#}, except that the additive 2-methyl-2,3-dihydrothieno[3,4-b][1,4]dioxane had a mass percent content of 6% instead of 1% and the resulting electrolyte was recorded as L4^{#}.

### Preparation of Lithium Ion Secondary Battery C4^{#}

This preparation method was the same as that of the lithium ion secondary battery C1^{#}, except that the electrolyte L4^{#} was used instead and the resulting lithium ion secondary battery was recorded as C4^{#}.

### Example 5

### Preparation of Electrolyte L5^{#}

This preparation method was the same as that of the electrolyte L1^{#}, except that the additive 2-methyl-2,3-dihydrothieno[3,4-b][1,4]dioxane had a mass percent content of 10% instead of 1% and the resulting electrolyte was recorded as L5^{#}.

### Preparation of Lithium Ion Secondary Battery C5^{#}

This preparation method was the same as that of the lithium ion secondary battery C1^{#}, except that the electrolyte L5^{#} was used instead and the resulting lithium ion secondary battery was recorded as C5^{#}.

### Example 6

### Preparation of Electrolyte L6^{#}

This preparation method was the same as that of the electrolyte L1^{#}, except that the additive 2-methyl-2,3-dihydrothieno[3,4-b][1,4]dioxane was replaced with the additive 3,4-propylenedioxythiophene (having a structural formula as shown by Formula IV, and abbreviated as Formula IV in Table 1) and the resulting electrolyte was recorded as L6^{#}.

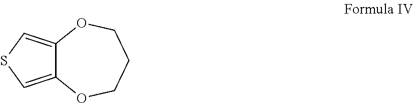

### Preparation of Lithium Ion Secondary Battery C6^{#}

This preparation method was the same as that of the lithium ion secondary battery C1^{#}, except that the electrolyte L6^{#} was used instead and the resulting lithium ion secondary battery was recorded as C6^{#}.

### Example 7

### Preparation of Electrolyte L7^{#}

This preparation method was the same as that of the electrolyte L2^{#}, except that the additive 2-methyl-2,3-dihydrothieno[3,4-b][1,4]dioxane was replaced with the additive 3,4-propylenedioxythiophene and the resulting electrolyte was recorded as L7^{#}.

### Preparation of Lithium Ion Secondary Battery C7^{#}

This preparation method was the same as that of the lithium ion secondary battery C1^{#}, except that the electrolyte L7^{#} was used instead and the resulting lithium ion secondary battery was recorded as C7^{#}.

### Example 8

### Preparation of Electrolyte L8^{#}

This preparation method was the same as that of the electrolyte L3^{#}, except that the additive 2-methyl-2,3-dihydrothieno[3,4-b][1,4]dioxane was replaced with the additive 3,4-propylenedioxythiophene and the resulting electrolyte was recorded as L8^{#}.

### Preparation of Lithium Ion Secondary Battery C8^{#}

This preparation method was the same as that of the lithium ion secondary battery C1^{#}, except that the electrolyte L8^{#} was used instead and the resulting lithium ion secondary battery was recorded as C8^{#}.

### Example 9

### Preparation of Electrolyte L9^{#}

This preparation method was the same as that of the electrolyte L4^{#}, except that the additive 2-methyl-2,3-dihydrothieno[3,4-b][1,4]dioxane was replaced with the additive 3,4-propylenedioxythiophene and the resulting electrolyte was recorded as L9^{#}.

### Preparation of Lithium Ion Secondary Battery C9^{#}

This preparation method was the same as that of the lithium ion secondary battery C1^{#}, except that the electrolyte L9^{#} was used instead and the resulting lithium ion secondary battery was recorded as C9^{#}.

### Example 10

### Preparation of Electrolyte L10^{#}

This preparation method was the same as that of the electrolyte L5^{#}, except that the additive 2-methyl-2,3-dihydrothieno[3,4-b][1,4]dioxane was replaced with the additive 3,4-propylenedioxythiophene and the resulting electrolyte was recorded as L10^{#}.

### Preparation of Lithium Ion Secondary Battery C10^{#}

This preparation method was the same as that of the lithium ion secondary battery C1^{#}, except that the electrolyte L10^{#} was used instead and the resulting lithium ion secondary battery was recorded as C10^{#}.

### Example 11

### Preparation of Electrolyte L11^{#}

This preparation method was the same as that of the electrolyte L1^{#}, except that the additive was replaced with a mixed system of 2-methyl-2,3-dihydrothieno[3,4-b][1,4]dioxane and vinylene carbonate (abbreviated as VC). 2-methyl-2,3-dihydrothieno[3,4-b][1,4]dioxane had a mass percent content of 1% and vinylene carbonate (VC) had a mass percent content of 0.1% in the electrolyte, and the resulting electrolyte was recorded as L11^{#}.

### Preparation of Lithium Ion Secondary Battery C11^{#}

This preparation method was the same as that of the lithium ion secondary battery C1^{#}, except that the electrolyte L11^{#} was used instead and the resulting lithium ion secondary battery was recorded as C11^{#}.

### Example 12

### Preparation of Electrolyte L12^{#}

This preparation method was the same as that of the electrolyte L1^{#}, except that the additive was replaced with a mixed system of 2-methyl-2,3-dihydrothieno[3,4-b][1,4]dioxane and vinylene carbonate (VC). 2-methyl-2,3-dihydrothieno[3,4-b][1,4]dioxane had a mass percent content of 1% and vinylene carbonate (VC) had a mass percent content of 1% in the electrolyte, and the resulting electrolyte was recorded as L12^{#}.

### Preparation of Lithium Ion Secondary Battery C12^{#}

This preparation method was the same as that of the lithium ion secondary battery C1^{#}, except that the electrolyte L12^{#} was used instead and the resulting lithium ion secondary battery was recorded as C12^{#}.

### Example 13

### Preparation of Electrolyte L13^{#}

This preparation method was the same as that of the electrolyte L1^{#}, except that the additive was replaced with a mixed system of 2-methyl-2,3-dihydrothieno[3,4-b][1,4]dioxane and vinylene carbonate (VC). 2-methyl-2,3-dihydrothieno[3,4-b][1,4]dioxane had a mass percent content of 1% and vinylene carbonate (VC) had a mass percent content of 3% in the electrolyte, and the resulting electrolyte was recorded as L13^{#}.

### Preparation of Lithium Ion Secondary Battery C13^{#}

This preparation method was the same as that of the lithium ion secondary battery C1^{#}, except that the electrolyte L13^{#} was used instead and the resulting lithium ion secondary battery was recorded as C13^{#}.

### Example 14

### Preparation of Electrolyte L14^{#}

This preparation method was the same as that of the electrolyte L1^{#}, except that the additive was replaced with a mixed system of 2-methyl-2,3-dihydrothieno[3,4-b][1,4]dioxane and vinylene carbonate (VC). 2-methyl-2,3-dihydrothieno[3,4-b][1,4]dioxane had a mass percent content of 1% and vinylene carbonate (VC) had a mass percent content of 6% in the electrolyte, and the resulting electrolyte was recorded as L14^{#}.

### Preparation of Lithium Ion Secondary Battery C14^{#}

This preparation method was the same as that of the lithium ion secondary battery C1^{#}, except that the electrolyte L14^{#} was used instead and the resulting lithium ion secondary battery was recorded as C14^{#}.

### Example 15

### Preparation of Electrolyte L15^{#}

This preparation method was the same as that of the electrolyte L1^{#}, except that the additive was replaced with a mixed system of 2-methyl-2,3-dihydrothieno[3,4-b][1,4]dioxane and vinylene carbonate (VC). 2-methyl-2,3-dihydrothieno[3,4-b][1,4]dioxane had a mass percent content of 1% and vinylene carbonate (VC) had a mass percent content of 10% in the electrolyte, and the resulting electrolyte was recorded as L15^{#}.

### Preparation of Lithium Ion Secondary Battery C15^{#}

This preparation method was the same as that of the lithium ion secondary battery C1^{#}, except that the electrolyte L15^{#} was used instead and the resulting lithium ion secondary battery was recorded as C15^{#}.

### Example 16

### Preparation of Electrolyte L16^{#}

This preparation method was the same as that of the electrolyte L11^{#}, except that 2-methyl-2,3-dihydrothieno[3,4-b][1,4]dioxane had a mass percent content of 0.1% and vinylene carbonate (VC) had a mass percent content of 1% in the electrolyte, and the resulting electrolyte was recorded as L16^{#}.

### Preparation of Lithium Ion Secondary Battery C16^{#}

This preparation method was the same as that of the lithium ion secondary battery C1^{#}, except that the electrolyte L16^{#} was used instead and the resulting lithium ion secondary battery was recorded as C16^{#}.

### Example 17

### Preparation of Electrolyte L17^{#}

This preparation method was the same as that of the electrolyte L11^{#}, except that 2-methyl-2,3-dihydrothieno[3,4-b][1,4]dioxane had a mass percent content of 3% and vinylene carbonate (VC) had a mass percent content of 1% in the electrolyte, and the resulting electrolyte was recorded as L17^{#}.

### Preparation of Lithium Ion Secondary Battery C17^{#}

This preparation method was the same as that of the lithium ion secondary battery C1^{#}, except that the electrolyte L17^{#} was used instead and the resulting lithium ion secondary battery was recorded as C17^{#}.

### Example 18

### Preparation of Electrolyte 18^{#}

This preparation method was the same as that of the electrolyte L11^{#}, except that 2-methyl-2,3-dihydrothieno[3,4-b][1,4]dioxane had a mass percent content of 6% and vinylene carbonate (VC) had a mass percent content of 1% in the electrolyte, and the resulting electrolyte was recorded as L18^{#}.

### Preparation of Lithium Ion Secondary Battery C18^{#}

This preparation method was the same as that of the lithium ion secondary battery C1^{#}, except that the electrolyte L18^{#} was used instead and the resulting lithium ion secondary battery was recorded as C18^{#}.

### Example 19

### Preparation of Electrolyte L19^{#}

This preparation method was the same as that of the electrolyte L11^{#}, except that 2-methyl-2,3-dihydrothieno[3,4-b][1,4]dioxane had a mass percent content of 10% and vinylene carbonate (VC) had a mass percent content of 1% in the electrolyte, and the resulting electrolyte was recorded as L19^{#}.

### Preparation of Lithium Ion Secondary Battery C19^{#}

This preparation method was the same as that of the lithium ion secondary battery C1^{#}, except that the electrolyte L19^{#} was used instead and the resulting lithium ion secondary battery was recorded as C19^{#}.

### Comparative Example 1

### Preparation of Electrolyte DL1^{#}

This preparation method was the same as that of the electrolyte L1^{#}, except that no additive was present in the electrolyte and the resulting electrolyte was recorded as DL1^{#}.

### Preparation of Lithium Ion Secondary Battery DC1^{#}

This preparation method was the same as that of the lithium ion secondary battery C1^{#}, except that the electrolyte DL1^{#} was used instead and the resulting lithium ion secondary battery was recorded as DC1^{#}.

### Comparative Example 2

### Preparation of Electrolyte DL2^{#}

This preparation method was the same as that of the electrolyte L12^{#}, except that only the additive vinylene carbonate was employed. Vinylene carbonate had a mass percent content of 1% in the electrolyte and the resulting electrolyte was recorded as DL2^{#}.

### Preparation of Lithium Ion Secondary Battery DC2^{#}

This preparation method was the same as that of the lithium ion secondary battery C1^{#}, except that the electrolyte DL2^{#} was used instead and the resulting lithium ion secondary battery was recorded as DC2^{#}.

The serial numbers and parameters of the batteries and electrolytes in Examples 1-19 and Comparative Examples 1-2 are as shown in Table 1.

TABLE 1

| | | | Composition of additive, and mass percent | | | |
|---|---|---|---|---|---|---|
| | Elec- | | content of each component in electrolyte | | | |
| | trolyte | Battery | Thiophene | | Film former of solid | |
| Examples | No. | No. | compounds | | electrolyte interface film | |
| Example 1 | L1^{#} | C1^{#} | 1%, | Formula III | / | |
| Example 2 | L2^{#} | C2^{#} | 0.1%, | Formula III | / | |
| Example 3 | L3^{#} | C3^{#} | 3%, | Formula III | / | |
| Example 4 | L4^{#} | C4^{#} | 6%, | Formula III | / | |
| Example 5 | L5^{#} | C5^{#} | 10%, | Formula III | / | |
| Example 6 | L6^{#} | C6^{#} | 1%, | Formula IV | / | |
| Example 7 | L7^{#} | C7^{#} | 0.1%, | Formula IV | / | |
| Example 8 | L8^{#} | C8^{#} | 3%, | Formula IV | / | |
| Example 9 | L9^{#} | C9^{#} | 6%, | Formula IV | / | |
| Example 10 | L10^{#} | C10^{#} | 10%, | Formula IV | / | |
| Example 11 | L11^{#} | C11^{#} | 1%, | Formula III | 0.1% | VC |
| Example 12 | L12^{#} | C12^{#} | 1%, | Formula III | 1% | VC |
| Example 13 | L13^{#} | C13^{#} | 1%, | Formula III | 3% | VC |
| Example 14 | L14^{#} | C14^{#} | 1%, | Formula III | 6% | VC |
| Example 15 | L15^{#} | C15^{#} | 1%, | Formula III | 10% | VC |
| Example 16 | L16^{#} | C16^{#} | 0.1%, | Formula III | 1% | VC |
| Example 17 | L17^{#} | C17^{#} | 3%, | Formula III | 1% | VC |
| Example 18 | L18^{#} | C18^{#} | 6%, | Formula III | 1% | VC |
| Example 19 | L19^{#} | C19^{#} | 10%, | Formula III | 1% | VC |

| Comparative | DL1^{#} | DC1^{#} | / | / | |
|---|---|---|---|---|---|
| Example 1 | | | | | |
| Comparative | DL2^{#} | DC2^{#} | / | 1% | VC |
| Example 2 | | | | | |

### Example 20

### Rate Discharge Performance Test of Lithium Ion Batteries

The lithium ion secondary batteries C1^{#}-C19^{#} prepared in Examples 1-19 and the lithium ion secondary batteries DC1^{#}-DC2^{#} prepared in Comparative Examples 1-2 were respectively subjected to a rate discharge performance test by the following specific method: the batteries were first charged to 4.35 V at a constant current of 0.5 C, then charged to a current of 0.05 C at a constant voltage of 4.35 V, left alone for 10 min, and then discharged to a cut-off voltage of 3.0 Vat a constant current of 0.2 C, 0.5 C, 1 C, 2 C and 3 C respectively. The discharge capacity was recorded and compared with a discharge capacity of 0.2 C to obtain the discharge efficiency at different discharge rates (15 batteries, the average value thereof was taken).

Retention ratio (%) of rate discharge capacity of lithium ion secondary battery=[rate discharge capacity/0.2 C rate discharge capacity]×100%

The test results of the batteries C1^{#}-C19^{#} and DC1^{#}-DC2^{#} are as shown in Table 2.

TABLE 2
Retention ratio of discharge capacity of batteries at different rates

| | Retention ratio (%) of discharge capacity | | | | |
|---|---|---|---|---|---|
| | at different discharge rates | | | | |
| Batteries | 0.2 C | 0.5 C | 1 C | 2 C | 3 C |
| C1^{#} | 100 | 98.5 | 97.5 | 88.5 | 75.3 |
| C2^{#} | 100 | 96.7 | 89.7 | 78.9 | 55.2 |
| C3^{#} | 100 | 98.3 | 96.9 | 85.6 | 74.8 |
| C4^{#} | 100 | 97.1 | 95.4 | 83.4 | 70.2 |
| C5^{#} | 100 | 96.5 | 89.5 | 78.1 | 55.4 |
| C6^{#} | 100 | 98.2 | 97.3 | 88.3 | 75.3 |
| C7^{#} | 100 | 96.4 | 88.5 | 77.5 | 54.4 |
| C8^{#} | 100 | 98.3 | 96.6 | 85.3 | 73.8 |
| C9^{#} | 100 | 97.0 | 95.0 | 83.0 | 70.2 |
| C10^{#} | 100 | 96.2 | 88.1 | 77.8 | 54.7 |
| DC1^{#} | 100 | 95.7 | 88.9 | 75.4 | 50.5 |
| C11^{#} | 100 | 99.1 | 97.2 | 93.0 | 85.4 |
| C13^{#} | 100 | 99.1 | 97.4 | 92.3 | 85.3 |
| C14^{#} | 100 | 98.4 | 88..9 | 75.7 | 69.7 |
| C15^{#} | 100 | 97.6 | 85.1 | 69.8 | 60.6 |
| C12^{#} | 100 | 99.5 | 97.5 | 93.5 | 85.3 |
| C16^{#} | 100 | 98.7 | 92.7 | 85.9 | 78.2 |
| C17^{#} | 100 | 99.2 | 96.9 | 92.6 | 84.8 |
| C18^{#} | 100 | 98.9 | 95.4 | 83.4 | 78.5 |
| C19^{#} | 100 | 98.5 | 92.5 | 81.1 | 65.4 |
| DC2^{#} | 100 | 97.2 | 90.4 | 80.1 | 55.9 |

It can be seen from Table 2 that the lithium ion batteries C1^{#}-C19^{#} in the technical solution of the present application have improved rate performance in terms of the retention ratio of discharge capacity at different rates as compared against DC1^{#} in which the electrolyte contains no additive. When the additive described in the present application is used in an electrolyte for a lithium ion battery, an interfacial film with good electrical conductivity is formed on an electrode surface in the cycle process of the battery, thereby facilitating the battery to obtain good rate performance.

Meanwhile, the amount of the additive also exerts some influence on the rate performance of the battery, i.e., both excessively low (0.1%) and excessively high (10%) concentrations achieve a limited effect in enhancing rate performance. The reasons are as follows: the film forming effect of an electrode surface is unobvious at an excessively low concentration (0.1%); however, an interfacial film formed on a positive electrode surface by the material may be thickened at an excessively high concentration (10%), thereby affecting lithium ion migration and resulting in poorer rate performance of the battery.

Based on the battery C1^{#}, vinylene carbonate (VC) of different masses was added to the C1^{#} battery electrolyte to obtain the batteries C10^{#}-C15^{#}; and based on the batteries C1-C5^{#}, vinylene carbonate (VC) having a mass percent content of 1% was added to obtain the batteries C12^{#} and C16^{#}-C19^{#}. The two batches of the VC-containing lithium ion batteries C10^{#}-C15^{#} as well as C12^{#} and C16^{#}-C19^{#} have enhanced rate performance, and especially, the retention ratio of discharge capacity at a high rate of 3 C thereof is far higher than that of the lithium ion battery C1^{#} in Example 1 and the lithium ion battery DC2^{#} in Comparative Example 2. However, the addition of vinylene carbonate (VC) having a higher concentration may degrade the rate performance of the lithium ion batteries, because an interfacial film formed on a negative electrode surface by the material may be thickened when vinylene carbonate (VC) has an excessively high concentration (the mass percent content generally exceeds 5%), thereby inhibiting lithium ion migration and resulting in poorer rate performance of the batteries.

The above results show that the use of the additive described in the present application obviously enhances the rate performance of traditional LiPF₆ batteries. When the additive is used in conjunction with a film former (vinylene carbonate) of a solid electrolyte interface film, the rate performance of the batteries is further enhanced.

### Example 21

### Internal DC Resistance Test of Lithium Ion Batteries

The lithium ion secondary batteries C1^{#}-C19^{#} prepared in Examples 1-19 and the lithium ion secondary batteries DC1^{#}-DC2^{#} prepared in Comparative Examples 1-2 were respectively subjected to an internal DC resistance (abbreviated as DCR) test by the following method:

At 25° C., the batteries were charged to 50% SOC at a constant current/voltage of 0.5 C (charged to 3.85 V at a constant current of 0.5 C, and then charged to 0.05 C at a constant voltage of 3.85 V), left alone for 10 min, discharged for 10 s at a constant current of 0.1 C (the voltage U₁ after discharging was recorded), and then discharged for 1 s at a constant current of 1 C (the voltage U₂ after discharging was recorded), wherein DCR=(U₁−U₂)/(1 C−0.1 C). The test data of the internal DC resistance (DCR) of the lithium ion batteries in the Example may be referred to Table 3.

TABLE 3
Internal DC resistance (DCR) values of lithium
ion batteries at 25° C. and 50% SOC

| | Internal DC resistance |
|---|---|
| Batteries | (mΩ) of batteries |
| C1^{#} | 42 |
| C2^{#} | 65 |
| C3^{#} | 43 |
| C4^{#} | 52 |
| C5^{#} | 68 |
| C6^{#} | 43 |
| C7^{#} | 67 |
| C8^{#} | 45 |
| C9^{#} | 54 |
| C10^{#} | 69 |
| DC1^{#} | 73 |
| C11^{#} | 54 |
| C13^{#} | 69 |
| C14^{#} | 75 |
| C15^{#} | 89 |
| C12^{#} | 52 |
| C16^{#} | 72 |
| C17^{#} | 51 |
| C18^{#} | 59 |
| C19^{#} | 76 |
| DC2^{#} | 92 |

It can be seen from Table 3 that the lithium ion batteries C1^{#}-C10^{#} in the technical solution of the present application have lower internal DC resistance as compared to DC1^{#} without any additive, indicating that interfacial films with good electrical conductivity are formed on electrode surfaces of the batteries C1^{#}-C10^{#}, and meanwhile, the concentration of the additive also exerts some influence on internal DC resistance (DCR), i.e., both excessively high and excessively low concentrations achieve a limited effect in enhancing internal DC resistance (DCR), because the film forming effect is unobvious at an excessively low concentration; however, an interfacial film formed on a positive electrode surface by the material may be thickened at an excessively high concentration, thereby affecting lithium ion migration and resulting in increased internal DC resistance (DCR) of the batteries.

Based on the battery C1^{#}, vinylene carbonate (VC) of different masses was added to the C1^{#} battery electrolyte to obtain the batteries C11^{#}-C15^{#}; and based on the batteries C1-C5^{#}, vinylene carbonate (VC) having a mass percent content of 1% was added to obtain the batteries C12^{#} and C16^{#}-C19^{#}. The two batches of the vinylene carbonate (VC)-containing batteries C11^{#}-C15^{#} as well as C12^{#} and C16^{#}-C19^{#} have impedance slightly higher than C1^{#}-C5^{#}, but still lower than DC2^{#}. It shows that, for a lithium ion battery using a thiophene compound and a film former of a solid electrolyte interface film, the internal resistance thereof is slightly higher than that of a battery using a thiophene compound alone, but still lower than that of a lithium ion battery using VC alone as an additive.

### Example 22

### Cycle Performance Test of Lithium Ion Batteries at 45° C.

At 45° C., the lithium ion secondary batteries C1^{#}-C19^{#} prepared in Examples 1-19 and the lithium ion secondary batteries DC1^{#}-DC2^{#} prepared in Comparative Examples 1-2 were charged to 4.35 V at a constant current of 1 C, then charged to a current of 0.05 C at a constant voltage, and then discharged to 3.0 V at a constant current of 1 C; charging/discharging was thus repeated; and the capacity retention ratio of the batteries after 50, 100, 200 and 300 cycles was respectively calculated. The cycle test data of the lithium ion batteries at 45° C. in the Example may be referred to in Table 4.

Capacity retention ratio (%) of lithium ion secondary battery after n cycles=[discharge capacity for the n^{th} cycle/discharge capacity for the first cycle]×100%.

TABLE 4
Test results of capacity retention ratio of lithium ion batteries
after repeated charging and discharging at 45° C.

| | Capacity retention ratio * (%) after n cycles at 45° C. | | | |
|---|---|---|---|---|
| Batteries | 50^{th} | 100^{th} | 200^{th} | 300^{th} |
| C1^{#} | 98.2 | 97.4 | 92.3 | 90.5 |
| C2^{#} | 98.1 | 96.4 | 89.4 | 80.2 |
| C3^{#} | 98.4 | 97.2 | 91.4 | 89.9 |
| C4^{#} | 97.5 | 92.5 | 88.5 | 85.9 |
| C5^{#} | 97.2 | 90.8 | 87.2 | 79.4 |
| C6^{#} | 98.0 | 97.3 | 92.1 | 90.2 |
| C7^{#} | 98.0 | 96.1 | 89.0 | 80.1 |
| C8^{#} | 98.1 | 97.0 | 91.3 | 89.5 |
| C9^{#} | 97.1 | 92.2 | 88.1 | 85.4 |
| C10^{#} | 97.2 | 90.7 | 87.1 | 79.2 |
| DC1^{#} | 97.5 | 90.1 | 82.5 | 72.4 |
| C11^{#} | 99.2 | 97.9 | 92.8 | 91.5 |
| C13^{#} | 99.1 | 97.9 | 92.7 | 91.6 |
| C14^{#} | 98.7 | 95.5 | 90.7 | 87.1 |
| C15^{#} | 97.2 | 91.8 | 87.2 | 80.8 |
| C12^{#} | 99.2 | 98.4 | 96.3 | 94.5 |
| C16^{#} | 98.7 | 97.4 | 92.4 | 85.2 |
| C17^{#} | 99.0 | 98.2 | 96.4 | 94.1 |
| C18^{#} | 98.4 | 96.5 | 91.5 | 88.9 |
| C19^{#} | 98.9 | 95.8 | 88.5 | 83.4 |
| DC2^{#} | 98.5 | 94.1 | 86.5 | 78.4 |

Note:
* charge-discharge rate is 1 C.

It can be seen from Table 4 that the capacity retention ratio of the lithium ion batteries C1^{#}-C10^{#} in the technical solution of the present application after cycling is obviously higher than that of the battery DC1^{#} without any additive. The cause of faster attenuation in a LiPF₆ battery free of any additive is that a LiPF₆ electrolyte continuously reacts with a positive electrode material, thereby resulting in reduced performance of the battery after long-term cycling. A thiophene compound as an additive added to the lithium ion batteries C1#-C10# is capable of forming a good interfacial film on a positive electrode surface to inhibit an electrolyte from reacting with a positive electrode material, and therefore the capacity retention ratio of the battery is still up to above 90% after C1# is cycled for 300 times. Meanwhile, the concentration of the additive also has some influence on capacity retention ratio, i.e. both excessively high and excessively low concentrations achieve a limited effect in enhancing cycle performance, because the performance enhancement is unobvious at an excessively low concentration; however, an interfacial film formed on a positive electrode surface is thick and the impedance of the system also gradually increases at an excessively high concentration, thereby resulting in faster attenuation in capacity.

Based on the battery C1^{#}, vinylene carbonate (VC) of different masses was added to the C1^{#} battery electrolyte to obtain the batteries C11^{#}-C15^{#}; and based on the batteries C1-C5^{#}, vinylene carbonate (VC) having a mass percent content of 1% was added to obtain the batteries C12^{#} and C16^{#}-C19^{#}. The two batches of the VC-containing lithium ion batteries C11^{#}-C15^{#} as well as C12^{#} and C16^{#}-C19^{#} have can maintain a higher capacity retention ratio. The comparison between the lithium ion batteries C11^{#}-C15^{#} using a thiophene compound and VC and the batteries C1^{#}-C5^{#} using a thiophene compound alone shows that the concentration of vinylene carbonate (VC) added has a greater influence, i.e. the capacity retention ratio of C11^{#}-C15^{#} is obviously higher that of C1^{#}-C5^{#} when the mass percent content of vinylene carbonate (VC) is lower than 5%, but on the contrary, a greater concentration of vinylene carbonate (VC) degrades the cycle performance of the batteries, because an interfacial film formed on a negative electrode surface may be thickened and the impedance of the system increases when vinylene carbonate (VC) has an excessively high concentration, thereby resulting in faster attenuation in capacity.

In summary, the lithium ion batteries in the technical solution of the present application have obviously enhanced comprehensive performance, which is mainly reflected in reduced DCR and improved rate performance and cycle performance.

Described above are merely several examples of the present application, and are not intended to limit the present application in any form. The present application is disclosed as above with reference to preferred examples, but these preferred examples are not intended to limit the present application. Variations or modifications made by any one skilled in the art using the above-disclosed technical content without departing from the scope of the technical solution of the present application are considered as equivalent embodiments and shall be covered within the scope of the technical solution.

What is claimed is:

1. An electrolyte additive, comprising a thiophene compound selected from at least one of a compound having a chemical structural formula as shown by Formula I and a compound having a chemical structural formula as shown by Formula II: 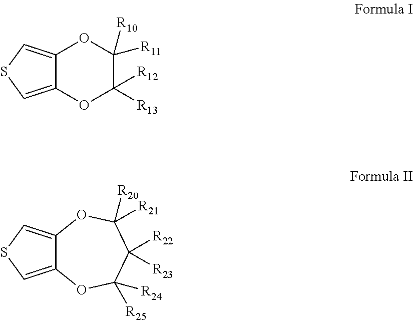
wherein R₁₀, R₁₁, R₁₂, R₁₃, R₂₀, R₂₁, R₂₂, R₂₃, R₂₄ and R₂₅ are respectively independently selected from hydrogen, fluoro, chloro, bromo, iodo, nitro, a sulfonic acid group, hydrocarbyl having 1-10 carbon atoms, and a group having 1-10 carbon atoms and containing at least one element selected from fluorine, chlorine, bromine, iodine, nitrogen, oxygen and sulfur.

2. The additive according to claim 1, wherein R₁₀, R₁₁, R₁₂, R₁₃, R₂₀, R₂₁, R₂₂, R₂₃, R₂₄ and R₂₅ are respectively independently selected from hydrogen, hydrocarbyl having 1-3 carbon atoms, and a group having 1-3 carbon atoms and containing fluorine and/or alkoxy.

3. The additive according to claim 1, wherein the thiophene compound is selected from at least one of 2-methyl-2,3-dihydrothieno[3,4-b][1,4]dioxane, 2-methoxymethyl-2,3-dihydrothieno[3,4-b][1,4]dioxane, 3,4-propylenedioxythiophene, 3,4-ethylenedioxythiophene, 3,4-(2,2-dimethylpropylenedioxy)thiophene and 3,4-(2,2-diethylpropylenedioxy)thiophene.

4. The additive according to claim 1, wherein the additive comprises a film former of a solid electrolyte interface film.

5. The additive according to claim 4, wherein the film former of a solid electrolyte interface film is selected from at least one of vinylene carbonate, fluoroethylene carbonate, chloroethylene carbonate, propane sultone, butane sultone and adiponitrile.

6. An electrolyte, comprising at least one of the additives according to claim 1.

7. The electrolyte according to claim 6, wherein the thiophene compound has a mass percent content of 0.1-10% in the electrolyte.

8. The electrolyte according to claim 6, wherein the thiophene compound has a mass percent content of 1%-8% in the electrolyte.

9. The electrolyte according to claim 6, wherein the film former of a solid electrolyte interface film has a mass percent content of 0.1-10% in the electrolyte.

10. A lithium ion battery, comprising at least one of the additives according to claim 1.
